(19) United States
(12) Patent Application Publication
(10) Pub. No.: US 2021/0304638 A1
(43) Pub. Date: Sep. 30, 2021

(54) Chicken Model for Robotic Basic Skills Training

(71) Applicant: Orsi Academy cvba, Melle (BE)

(72) Inventors: Alexandre Mottrie, Aalst (BE); Stefano Puliatti, Modena (IT); Marco Amato, Modena (IT); Anthony Gallagher, Newtownabbey (GB)

(21) Appl. No.: 17/332,651

(22) Filed: May 27, 2021

Related U.S. Application Data
(63) Continuation-in-part of application No. 17/273,006, filed on Mar. 3, 2021.
(60) Provisional application No. 62/726,669, filed on Sep. 4, 2018.

(51) Int. Cl. G09B 23/28; A61B 34/30; B25J 9/16

(52) U.S. Cl. CPC G09B 23/28; B25J 9/1679; A61B 34/30

(57) ABSTRACT

The present invention provides a chicken-based model for simulation training using observable, explicitly defined and validated performance metrics that appropriately characterize the skill to be trained, e.g., suturing, knotting, dissecting and coagulating. The simulation-based training gives trainees precise feedback on their performances with specific recommendations for improvement proximate to the performance, according to the use of deliberate practice in proficiency-based progression (PBP) methodology. Trainees are also provided a quantitative performance benchmark (for each task) to work toward that provides a valid representation of their skill level on the specific task. The trainees must demonstrate the ability to meet specific performance benchmarks before they are allowed to progress in their training program.

### CROSS-REFERENCE TO RELATED APPLICATIONS

This application is a continuation-in-part of U.S. patent application Ser. No. 17/273,006, filed on Mar. 3, 2021, which is a national stage application filed under 35 USC § 371 of International Patent Application No. PCT/IB2019/057426, filed on Sep. 3, 2019, which claims priority to U.S. provisional application 62/726,669, filed on Sep. 4, 2018, all of which are incorporated by reference herein in their entireties.

### BACKGROUND OF THE INVENTION

Since its introduction in 1999, robot-assisted surgery has shown many advantages over traditional approaches including ten times magnified three-dimensional (3D) view of the surgical field, shortening the learning curve and operative times, and more precise surgical movements thanks to the implementation of endo-wrist mechanisms. However, robotic surgeons and surgical societies agree that there is a deficiency of structured robotic training curricula and objective parameters that trainees must achieve before transitioning to the operating room.

Therefore, a surgical training program based on objective, fair, transparent and validated performance metrics is needed. This goal has been made more difficult by the development and release on the market of various surgical robotic platforms competing with the currently leading system, the DaVinci surgical system, each with its own hardware, operation, and features.

### SUMMARY OF THE INVENTION

The proficiency-based progression (PBP) training methodology has shown, in several prospective, randomized, and double-blind studies, the capability to reduce the number of performance errors by ˜60% and to improve clinical outcomes. Standardized and validated surgery curricula for training of basic surgical skills are a crucial cornerstone of standardization of training, accreditation, and certification of surgeons for robotic surgery.

Standardized and validated surgery curricula have been previously described in International Publication No. WO 2020/049464, entitled “Simulator Based Training Processes for Robotic Surgeries,” assigned to the current applicant and hereby incorporated by reference in its entirety.

The scientific community aims to develop training methodologies that allow surgical activity on human patients only after demonstrating a defined performance benchmark in the use of the robot (basic device training) and in the acquisition of surgical skills (basic surgical skills). Suturing, knotting, coagulating, and dissecting have been identified as the four fundamental basic surgical skills that the trainee robotic surgeon must acquire before being allowed to continue with the learning of an entire robotic procedure. The use of a non-living chicken for surgical training has been found to be a good model for the training of basic robotic skills.

The present invention provides a chicken-based model for simulation training using validated metrics that appropriately characterize the skill to be trained, e.g., suturing, knotting, dissecting, and coagulating. The simulation-based training gives trainees appropriate and precise feedback on their performances with specific recommendations for improvement proximate to the performance, according to the use of deliberate practice in PBP methodology. Trainees are also provided a quantitative performance benchmark to work towards that provides a valid representation of their skill level in a certain skill. The trainees must demonstrate the ability to meet specific performance benchmarks before they can progress in their training program.

In one embodiment of the invention, a method of training a trainee for robotic basic surgical skills on a chicken model is provided, consisting of: (a) recording a video of the trainee performing a task on the chicken model; (b) reviewing the video of the trainee performing the task on the chicken model; (c) determining whether or not the procedure has been performed according to validated metrics, where the metrics are at least one of a distinct performance component, a sequence in which specific steps should be completed, and an indication on instruments use; (d) inputting a first indication if the metric is performed and a second indication if the metric is not satisfied, into an evaluation form; (e) providing a summary report based on the evaluation report form of the trainee's performance.

It is thus a feature of at least one embodiment of the invention to allow a trainee to receive early feedback on basic surgical skills performed on a chicken model prior to learning an entire surgical procedure.

The trainee should perform the procedure using controls manipulating the corresponding robotic arms of a robot interacting with the chicken model.

It is thus a feature of at least one embodiment of the invention to closely simulate procedures performed on a human patient using a non-living chicken model.

For robotic suturing and knot tying skills training, the preparation of the chicken model is standardized as well as the robot positioning. The chicken is provided after removing all abdominal organs, except for 6-8 cm of the cloaca (starting measurements from the anus) and the stomach.

The set of metrics for suturing and knotting skills training include at least one of the following: suturing of a posterior wall; suturing of a left lateral wall; suturing of a right lateral wall; suturing of an anterior wall; and knotting.

The set of metrics for suturing and knotting skills training include deviations from optimal performance. The deviation may be an error including at least one of: conflict of instruments; missed grasp; instrument not assisting; tear or injury of tissue; damage of the suture; incorrect needle grasp; excessive manipulation, incorrect tip grasp; incorrect bite; incomplete or repeated bite; incorrect suture bite; needle out of view; missed loop; tailed loop; loose knot; missed double overhand knot; failure to alternate the direction of the last two throws; and failure to progress.

The deviation may also be a critical error including at least one of: an anastomosis leakage; a broken needle or suture; and a catheter fixation during anastomosis.

The summary report may include an average score for steps of the procedure. The summary report may include total time to perform the procedure. The time limit for procedure completion of suturing and knotting skills training is 40 minutes.

It is thus a feature of at least one embodiment of the invention to provide trainee feedback for the suturing and knotting skills training task that includes an assessment on the performance of specific steps or tasks as well as noting errors and critical errors during steps or tasks.

For robotic dissection skill training, the preparation of the chicken model is standardized as well as the robot positioning. The chicken is fixed to a tray with tape, with the right leg (left leg for left-handers) dislocated and stretched perpendicular to the body.

The set of metrics of the dissection skill training task include at least one of the following: initial skin incision; skin incision development; dissection of the upper leg; and dissection of the lower leg.

The set of metrics of the dissection skill training task also includes deviations from optimal performance. The deviation may be an error including at least one of: conflict of instrument; missed grasp; second arm instrument not assisting; fourth arm instrument not assisting; incorrect assistance of the second or fourth arm; incorrect use of the EndoWrist (for the second and fourth arm); drop of the skin traction; grasping of the skin (except for the initial incision); tear, burning or damage the skin; grasping of the muscle; tear, burning or damage of the muscle; skin incision outside marking lines; camera off target; instrument out of view; and fat left on the muscle.

The deviation may also be a critical error including at least one of: a tear resulting in a hole in the skin of the lower leg; and exceeded a time limit.

The summary report may include an average score for steps of the procedure. The summary report may include total time to perform the procedure. The time limit for procedure completion of robotic skin dissection task is 15 minutes.

It is thus a feature of at least one embodiment of the invention to provide trainee feedback for the dissection skills training task that includes an assessment on the performance of specific steps or tasks as well as noting errors and critical errors during steps or tasks.

For robotic dissection and coagulation skills training, the preparation of the chicken model is standardized as well as the robot positioning. The chicken is fixed to a tray with tape, the chicken laying on the breast and the right leg (left leg for left-handers) folded against the body.

The set of metrics for the dissection and coagulation skill training include: skin incision with the use of diathermic energy; dissection between the muscle's body; dissection of the vessel (>3 cm) positioned above and posteriorly to the bone; positioning and clipping of the vessel-loop; double (one per side) clipping of the vessel; bipolar coagulation of the vessel in between of the clips; and vessel cutting in between the coagulated tissue.

The set of metrics for the dissection and coagulation skill training includes deviation from optimal performance. The deviation may be an error including at least one of: conflict of instrument; missed grasp; second arm instrument not assisting; fourth arm instrument not assisting; tear, burning or damage of the muscle; grasping of the muscle; tear, burning or damage of the skin; skin incision outside marking lines; camera off target; grasping of the vessel; collision with the assistant instrument; incorrect exposure of the vessel for the double clipping; breaking or repositioning of the vessel-loop; bipolar coagulation of the surrounding tissue; excessive squeezing of the bipolar jaws which causes suboptimal coagulation; and instrument out of view.

The deviation may also be a critical error including at least one of: a blood vessel rupture during dissection or bipolar coagulation; exceeding a time limit for the vessel identification; and exceeding a time limit for the entire exercise.

The summary report may include an average score for steps of the procedure. The summary report may include total time to perform the procedure. The time limit for procedure completion of robotic skin dissection task is 15 minutes.

It is thus a feature of at least one embodiment of the invention to provide trainee feedback for the dissection and coagulation skills training task that includes an assessment on the performance of specific steps or tasks as well as noting errors and critical errors during steps or tasks.

These and other objects, advantages, and features of the invention will become apparent to those skilled in the art from the detailed description and the accompanying drawings. It should be understood, however, that the detailed description and accompanying drawings, while indicating preferred embodiments of the present invention, are given by way of illustration and not of limitation. Many changes and modifications may be made within the scope of the present invention without departing from the spirit thereof, and the invention includes all such modifications.

### BRIEF DESCRIPTION OF THE FIGURES

The patent or application file contains at least one drawing executed in color. Copies of this patent or patent application publication with color drawing(s) will be provided by the Office upon request and payment of the necessary fee.

A clear conception of the advantages and features constituting the present invention, and of the construction and operation of typical mechanisms provided with the present invention, will become more readily apparent by referring to the exemplary, and therefore non-limiting, embodiments illustrated in the drawings accompanying and forming a part of this specification, wherein like reference numerals designate the same elements in the several views, and in which:

FIG. 1 is a simplified top plan view of a trainee performing a robotic-assisted surgical procedure according to the present invention providing multiple video cameras recording the trainee's steps, with assistance from an anesthesiologist, nurse, and surgical assistant;

FIG. 2 is a simplified diagram of an assessment tool according to the present invention, including the recording of video images and scoring by a remote expert;

FIG. 3 is an example of a procedural evaluation data scoresheet (a portion shown), represented here as a logical table with different testing metrics organized into a plurality of phases;

FIG. 4 is an example summary report generated using inputs taken from a procedural evaluation data scoresheet of FIG. 3 and providing feedback on the trainee's performance;

FIG. 5 is a flowchart of the method performed during the evaluation of the trainee's performance; and

FIG. 6 is a data scoresheet for a suturing and knot tying task on a chicken model of the present invention;

FIG. 7 is a data scoresheet for a skin dissection task on the chicken model of the present invention;

FIG. 8 is a data scoresheet for a dissection and coagulation task on the chicken model of the present invention;

FIG. 9 shows an inside view of the chicken model and showing the required organs for the suturing and knot tying task of FIG. 6 with the chicken model;

FIG. 10 shows removal of lower and upper anterior abdominal wall of the chicken model for preparation of the suturing and knot tying task of FIG. 6 with the chicken model;

FIG. 11 shows an outside view of the chicken model after preparation for the suturing and knot tying task of FIG. 6 with the chicken model;

FIG. 12 shows a distance between a camera trocar and the target for the suturing and knot tying task of FIG. 6 on the chicken model (18-20 cm);

FIG. 13 shows a distance between trocars for the suturing and knot tying task of FIG. 6 on the chicken model (8-10 cm);

FIG. 14 shows needles and sutures used for the suturing and knot tying task of FIG. 6 on the chicken model (two Polysorb 3/0 sutures of 10 cm each);

FIG. 15 shows a first step of the suturing and knot tying task of FIG. 6 on the chicken model (posterior wall);

FIG. 16 shows a second step of the suturing and knot tying task of FIG. 6 on the chicken model (left lateral wall);

FIG. 17 shows a third step of the suturing and knot tying task of FIG. 6 on the chicken model (right lateral wall);

FIG. 18 shows a fourth step of the suturing and knot tying task of FIG. 6 on the chicken model (anterior wall);

FIG. 19 shows a fifth step of the suturing and knot tying task of FIG. 6 on the chicken model (knotting);

FIG. 20 shows an outside view of the chicken model after preparation for the skin dissection task of FIG. 7;

FIG. 21 shows a division of upper and lower chicken model leg according to metrics of the skin dissection task of FIG. 7;

FIG. 22 shows lateral limits of dissection marked by stitches for the skin dissection task of FIG. 7 on the chicken model;

FIG. 23 shows camera and operative trocars disposition for the skin dissection task of FIG. 7 on the chicken model;

FIG. 24 shows a first step of the skin dissection task of FIG. 7 on the chicken model (initial skin incision);

FIG. 25 shows a second step of the skin dissection task of FIG. 7 on the chicken model (development of the skin incision);

FIG. 26 shows a third step of the skin dissection task of FIG. 7 on the chicken model (dissection of the upper leg);

FIG. 27 shows a fourth step of the skin dissection task of FIG. 7 on the chicken model (dissection of the lower leg);

FIG. 28 shows an outside view of the chicken model after preparation for the dissection and coagulation task of FIG. 8;

FIG. 29 shows a first step of dissection and coagulation task of FIG. 8 on the chicken model (initial skin incision);

FIG. 30 shows a second step of dissection and coagulation task of FIG. 8 on the chicken model (dissection between the leg's muscles' body);

FIG. 31 shows a third step of dissection and coagulation task of FIG. 8 on the chicken model (dissection of the vessel >3 cm);

FIG. 32 shows a fourth step of dissection and coagulation task of FIG. 8 on the chicken model (positioning and clipping of the vessel loop);

FIG. 33 shows a fifth step of dissection and coagulation task of FIG. 8 on the chicken model (double clipping of the vessel);

FIG. 34 shows a sixth step of dissection and coagulation task of FIG. 8 on the chicken model (bipolar coagulation of the vessel in between the clips); and

FIG. 35 shows a seventh step of dissection and coagulation task of FIG. 8 on the chicken model (cutting the vessel in between of the clips and in the middle of the coagulated tissue).

In describing the embodiment of the invention, which is illustrated in the drawings, specific terminology will be resorted to for the sake of clarity. However, it is not intended that the invention be limited to the specific terms so selected, and it is to be understood that each specific term includes all technical equivalents, which operate in a similar manner to accomplish a similar purpose. For example, the words “connected”, “attached”, or terms similar thereto are often used. They are not limited to direct connection but include connection through other elements where such connection is recognized as being equivalent by those skilled in the art.

### DETAILED DESCRIPTION OF THE INVENTION

The various feature and details of the subject matter disclosed herein are explained more fully in detail in the following description.

Skilled Performance

Skill can be defined as “completing a task as a skilled individual does”. However, this definition does not provide a testable model of skilled performance that could be used to properly define and score a performance. Several psychologists have tried to characterize the concept of “skill”, applying a detailed task analysis to it and then defining the fundamental aspects of performance that constitute the building blocks of a skill.

Once a task analysis is performed, it is important to quantitatively validate whether the aforementioned characterization fits with what is known about the skill. The task-analysis derived characterizations of skill should allow for ordinal differentiation among different levels of performance.

Performance Metrics

According to PBP methodology, the entire process of metric development starts by analyzing a certain skill through a detailed task analysis. Task analysis is performed to identify salient performance characteristics to train and assess and then reach a consensus between experts on the characteristics of the reference procedure performed by practicing procedure experts.

Procedure performance may be guided by manufacturer recommendations on device usage, scientific society guidelines, and results from empirical studies. In the absence of a consensus between the experts on the aforementioned items, practical clinical procedure/performance wisdom may be employed.

Task analysis involves a breakdown of the skills itself, identifying the steps necessary to complete the procedure. Each step needs to be operationally defined, specifying order, duration and results of the step, rather than simply described.

The units of performance identified are defined as metric units of task execution, providing a quantitative standard of measurement. These units are used to define and shape the simulations models developed to train a task performance.

Metrics definitions should be complete, indicating the beginning and endpoints of each step, and with enough detail to score performance reliably.

Metrics should define errors for each procedure step. Errors are defined as actions that deviate from optimal practice, while critical errors are unsafe actions which may lead to bad outcomes.

Finally, metrics should be scored as a binary outcome (yes or no), rather than in a Likert-scale fashion.

After their development, metrics must be validated. Metrics are first validated according to content validity. Content validity consists of an evaluation of the contents of metric units by a panel of experts. Once definitions are verified, metrics must undergo construct validation. The aim of construct validity of metrics is to allow an effective differentiation between different levels of performance. Moreover, construct validity will guide the skills benchmarking process, to define the proficiency level which trainees should acquire before moving further in their training process.

Task Execution and Performance Assessment

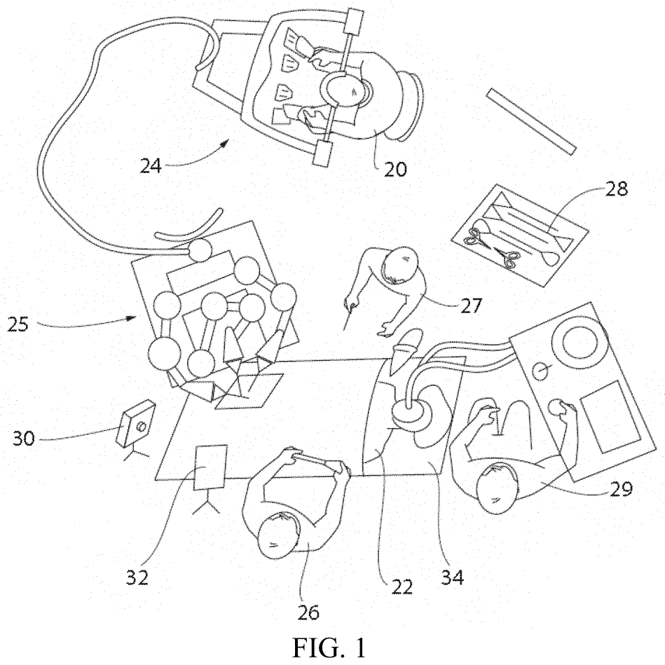

Referring initially to FIG. 1, a trainee 20 is instructed to perform a predetermined surgical technique, e.g., basic robotic skills training on chicken model, using robot controls 24 mechanically manipulating corresponding robotic arms 25. The robotic arms 25 are generally smaller than the robot controls 24. The surgical technique is performed on a simulation model, animal model or cadaver 22 facilitating performance evaluation. The trainee 20 may be instructed to demonstrate and complete all of the steps for the surgical task that they would normally perform in clinical practice on a real patient.

During skills assessment, coaching of the trainee 20 is not allowed although the trainee 20 may be assisted by a surgical assistant 26 who may only act at the specific direction or instruction of the trainee 20. The skills assessment may also involve the participation of a nurse 27 and/or anesthesiologist 29, who would be present during a real-life procedure and may also serve as a supervising medical professional to the trainee 20. The trainee 20 is provided all standard robotic and surgical instruments 28 necessary to complete the surgical task.

In the illustrated and described embodiment, the surgical technique may be surgical task, although it is understood that the present invention also contemplates other robotic surgical procedures. For example, the trainee 20 may be instructed to establish portals (for the robot 24) or complete a thorough docking of the robot 24.

A continuous video recording may be made simultaneously with one or more cameras, for example, two cameras 30, 32, situated within the operating room. The recording may record the surgery from the beginning of the procedure, with an external camera view of the surgical area while positioning of the simulation model, animal model or cadaver 22 on the operating table 34, and continue with the first endoscopic view of the surgical area during the insertion of the robotic instruments 24, and end with the withdrawal of the endoscope after the trainee's examination of the completed surgical task. There may be multiple cameras 30, 32 and the cameras 30, 32 may capture different angles or perspectives of the procedure. The video recordings are live streamed or stored in archive for expert 36 evaluation, to be further described below.

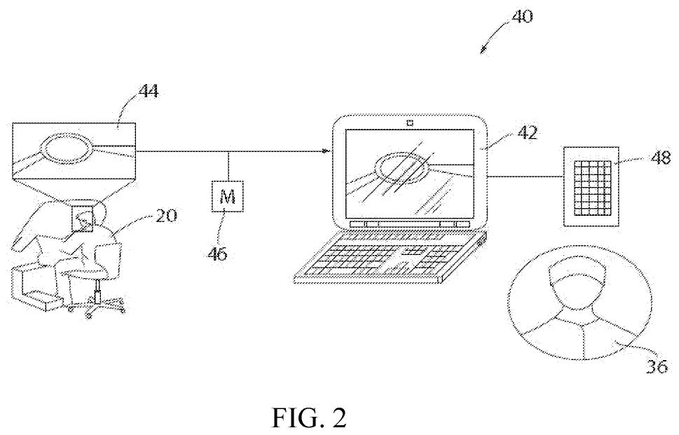

Referring now to FIG. 2, an assessment tool 40 for trainee performance scoring may provide video display monitors 42 for displaying video images of the type acquired by the cameras 30, 32 during the robotic simulator model testing (described above). The video display monitor or monitors 42 may be in a separate room from the testing or otherwise in a remote location. The video images 44 may be played in real time and evaluated by the expert 36 or may be stored in memory 46 so that the expert 36 may play back the video images 44 and review the video images 44 as many times as necessary to properly view the trainee's performance. A computer monitor 42 may provide the case to be reviewed by the expert 36 and an evaluation report input into which procedural evaluation data scoresheet 48 may be entered by the expert 36.

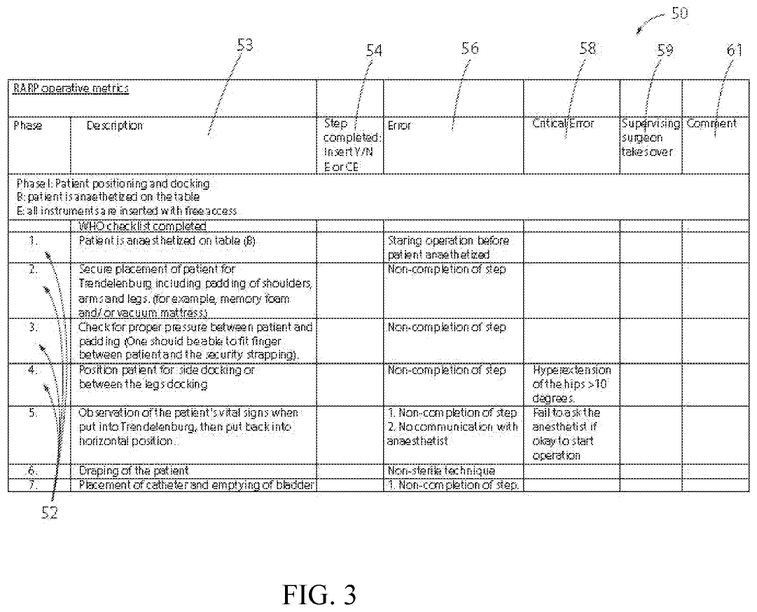

Referring now to FIG. 3, the procedural evaluation data scoresheet 48 may be represented as a logical table 50 (a portion of the full scoresheet 48 is shown) listing different testing metrics 53 (represented by rows) and tying them to evaluation scores organized into different sub-phases 52 of the procedure as either occurring or not occurring 54, as evaluated by the expert 36. The sub-phases 52 may be added together and averaged to provide an average total step and total error score for each phase.

Metrics 53 may include discrete performance elements (steps), the order in which specific operative steps should be accomplished, and/or the instruments and the manner in which they should be used. Metrics may also include deviations from optimal performance that should be avoided also known as errors 56. Additional metrics may include special designations for more serious or critical errors 58 defined by events that, by themselves, could either jeopardize the outcome of the procedure or lead to iatrogenic damage to the internal organs. Optionally, the expert 36 may provide a column 59 to indicate if a supervising surgeon had to take over for some or all of the procedure, which may indicate a failed performance element. The procedural evaluation data scoresheet 48 may also provide a column for comments 61 during each performance element, which may be positive or negative and may be used to help the trainee 20 improve their performance.

In order to maintain consistency in evaluation it may be desired to apply the convention that an event must be observed to be scored. For such metrics, the metric is scored in binary fashion, for example, as either yes (1) or no (0), or occurring (1) or not occurring (0). In an alternative embodiment other scoring indications, which are not binary in nature, may optionally be used such as colors or shapes denoting a particular score value.

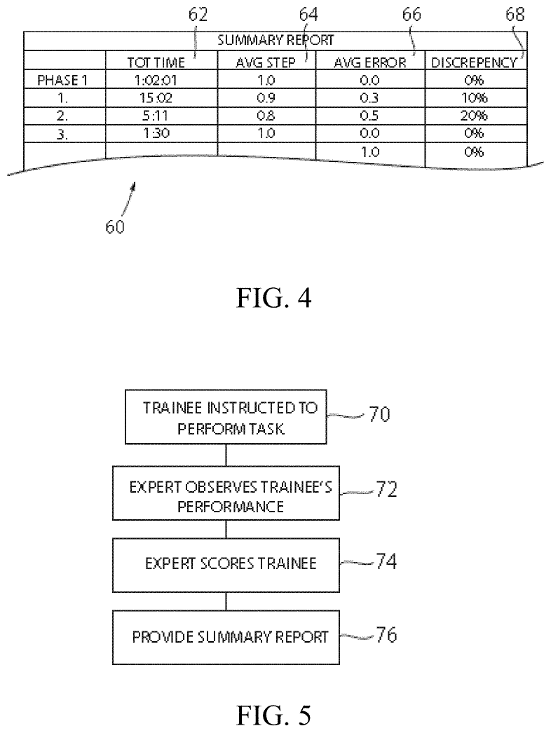

Referring to FIG. 4, following input of the procedural evaluation data scoresheet 48, the simulation may be programmed to provide the trainee 20 with a summary report 60 of their performance, e.g., related to performance of the surgical task, and accurate feedback based upon the procedural evaluation data scoresheet 48. The summary report may be provided on the computer monitor 42 or in a paper printout. The total time in minutes 62 taken by the trainee 20 to perform the procedural components in each video may be provided in the summary report 60.

The evaluation scores for multiple, for example, two, experts 36 evaluating the same trainee may be added together and averaged to provide average scores, namely, an average step score 64 and an average error score 66 provided in the summary report 60 for each phase. The summary report 60 may also contain average scores for each sub-phase of the procedure. An indication of discrepancy between different experts' scores may also be indicated in the summary report 60 and reported as an inter-rater reliability score 68.

Referring to FIG. 5, a method of training the trainee 20 on a surgical/procedural technique is represented by the flow chart shown. The trainee 20 is instructed to perform a specific surgical task on the training tool or simulation as represented by step 70. The task may include a basic robotic skills training task or another robotic surgery procedure. The specific surgical task is associated with a list of detailed metrics, defining the proper steps to be performed during the task and the unwanted errors commonly encountered during the task. The metrics are generally unique to each specific surgical task.

The expert 36 observes the trainee's performance in person or in a recording of the performance that is played back as represented by step 72. The expert 36 then uses an assessment tool to score the trainee 20 on the variety of metrics as represented by step 74. The assessment asks whether the metric (step or error) occurs or does not occur during the trainee's surgical performance of the task, thus, minimizing any review bias occurring in a more qualitative analysis.

Once the expert 36 has completed the assessment, the scores of all the experts are calculated to provide the trainee 20 summary report 60 with performance scores and other evaluation information (such as expert notes) as represented by step 76. The scores provide detailed and specific feedback to the trainee 20 in a timely manner, close in time to the performance of the task. Based upon the trainee's score, the trainee 20 may use the feedback to improve their performance and if successful may advance to a higher stage of training. The specificity of the scores allows the trainee 20 to practice specific metrics and aspects of the task for a more deliberate training method.

### Example

Basic Robotic Skills Training on a Chicken Model

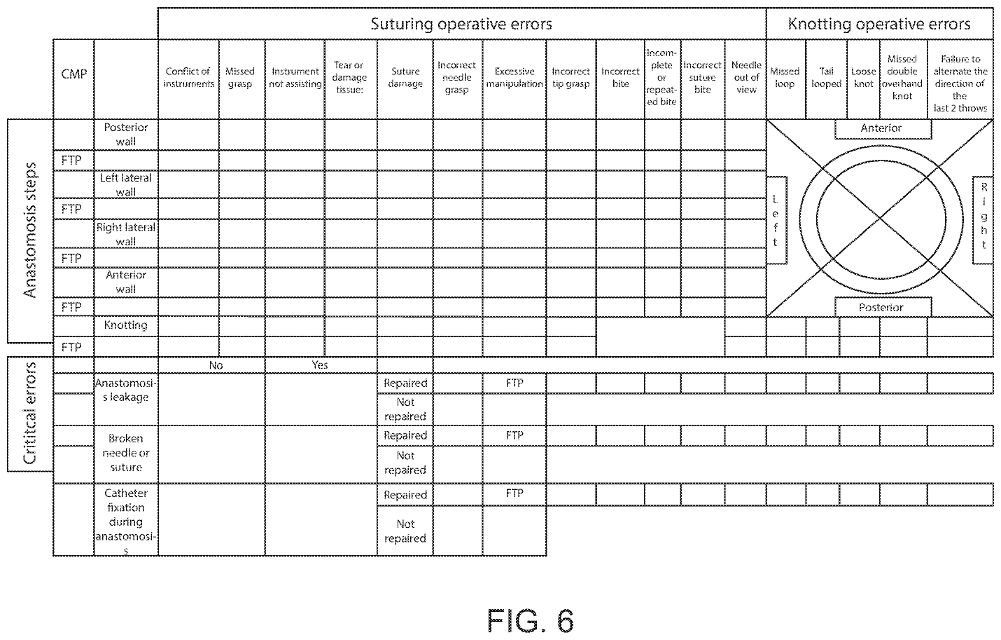

Trainees are instructed to perform robotic skill tasks using robot controls mechanically in order to manipulate the corresponding robotic arms. The surgical tasks are performed on a non-living chicken model. During skills assessment, coaching of the trainee is not allowed. The trainee is provided all standard robotic and surgical instruments necessary to complete the tasks. The procedural evaluation data scoresheets are represented as logical tables (FIGS. 6, 7 and 8), listing different testing metrics.

1. Suturing and Knotting Task

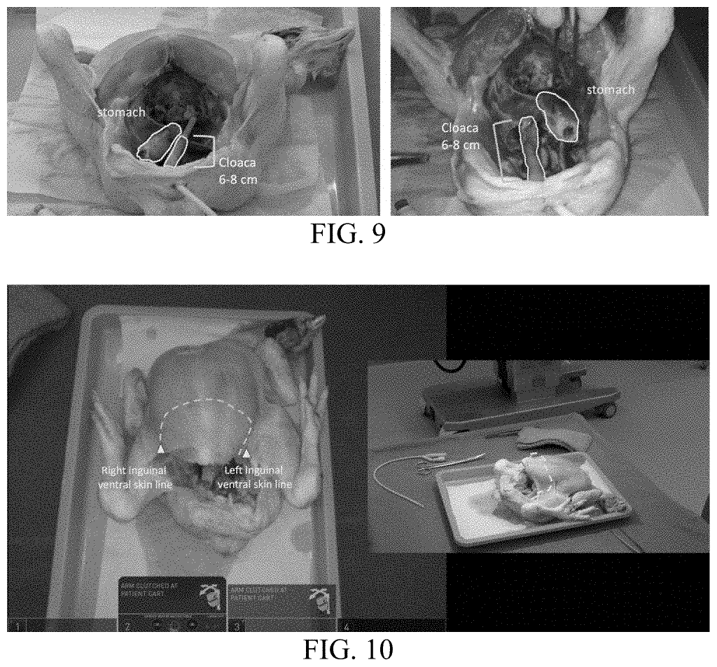

The preparation of the chicken model is standardized. The chicken is provided after removing all abdominal organs, except for 6-8 cm of the cloaca (starting measurements from the anus) and the stomach. Ideally, the cloaca overlaps the stomach for 1 cm. Shorter or longer overlap may impede the correct execution of the urethro-vesical anastomosis training (FIG. 9).

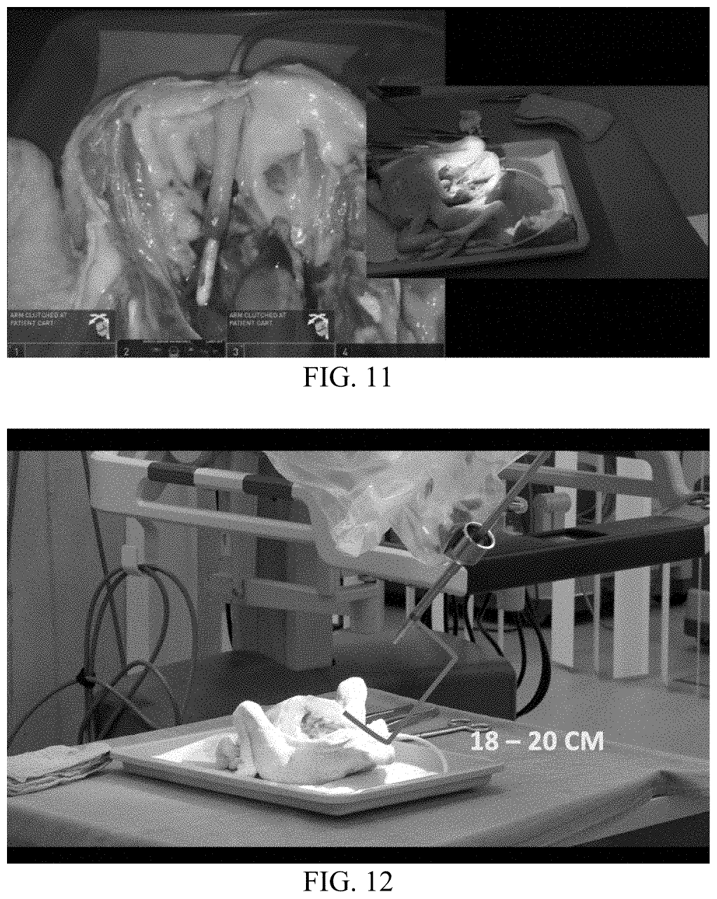

The preparation of the chicken starts by opening the chicken legs, followed by cutting and removing the lower and upper anterior abdominal walls (FIG. 10). A 12Ch Foley latex catheter is placed in the cloaca (FIG. 11).

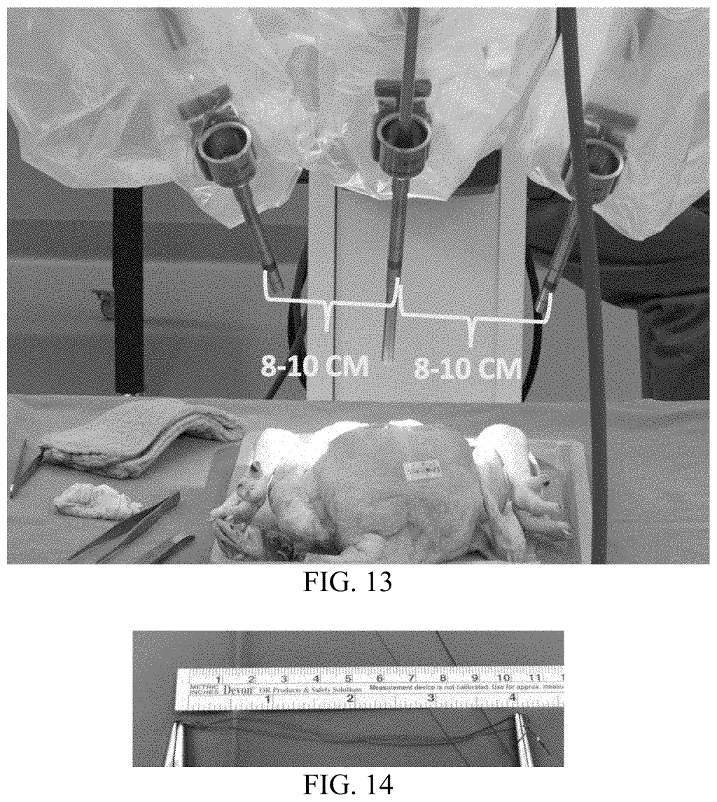

The suturing and knot tying task on the chicken model can be completed with any surgical robot model currently available on the market. The robot positioning is standardized. The table where the chicken model is placed should be fixed. The patient cart is positioned to maintain the camera trocar at a distance of 18-20 cm from the target (FIG. 12). Robotic trocars must be positioned at a distance of 8-10 cm apart (FIG. 13). Two large needle drivers and one Prograsp™ forceps are used. Two Polysorb™ sutures of 10 cm each, knotted at the end with four knots, are used to perform the task (FIG. 14). The anastomosis is executed according to the technique described by Roland F. Van Velthoven.

Referring to FIG. 6, the suturing and knotting task metrics are grouped into five separate steps of the procedure.

The metrics contain a series of related, unambiguously defined, observable procedure steps with specific beginning and ending points to be evaluated. There are also errors and critical errors for the expert to assess throughout the whole procedure. The scoring of the errors starts when the trainee engages the first bite on the stomach. Suturing and knotting task include five steps, 66 errors (5 unique) and 3 critical errors. The five steps and the errors and critical errors associated with the five steps are listed in the table of FIG. 6, and as further described below.

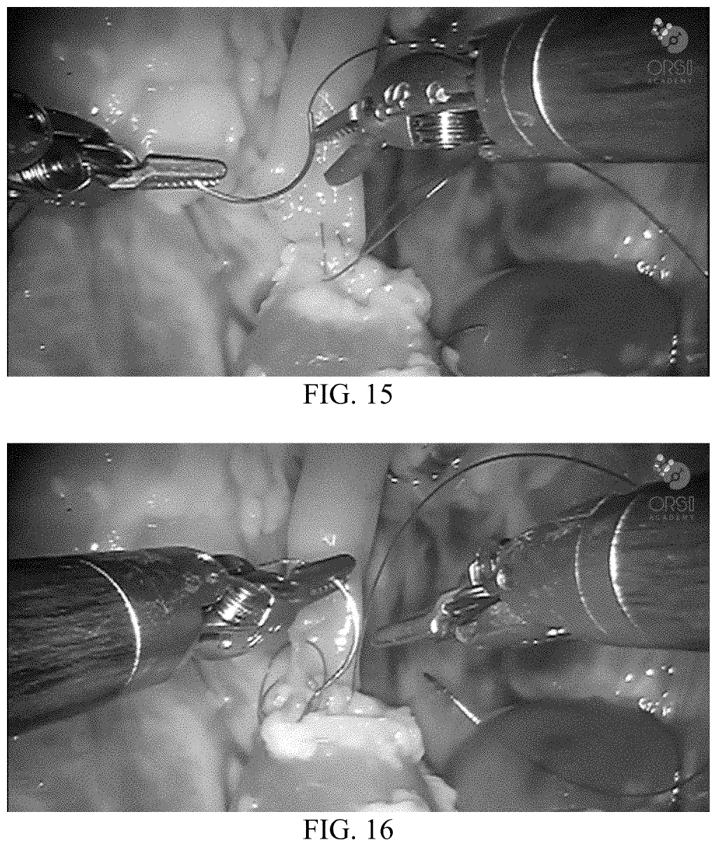

The first step includes the first four needle bites between the stomach and the cloaca. The needle direction should be from outside to inside when passing through the stomach and from inside to outside when biting the cloaca (FIG. 15). The first step has the associated errors of (1) conflicts of instruments, (2) missed grasp, (3) instrument not holding the suture nor assisting, (4) tear or injury of the tissue, (5) damage of the suture, (6) incorrect needle grasp, (7) excessive manipulation of the needle, (8) incorrect tip grasp, (9) incorrect bite, (10) incomplete or repeated bite, (11) incorrect suture bite, (12) needle out of view, (13) fail to progress in that specific step of the anastomosis and critical errors of (1) broken needle or suture and (2) catheter fixation during the anastomosis.

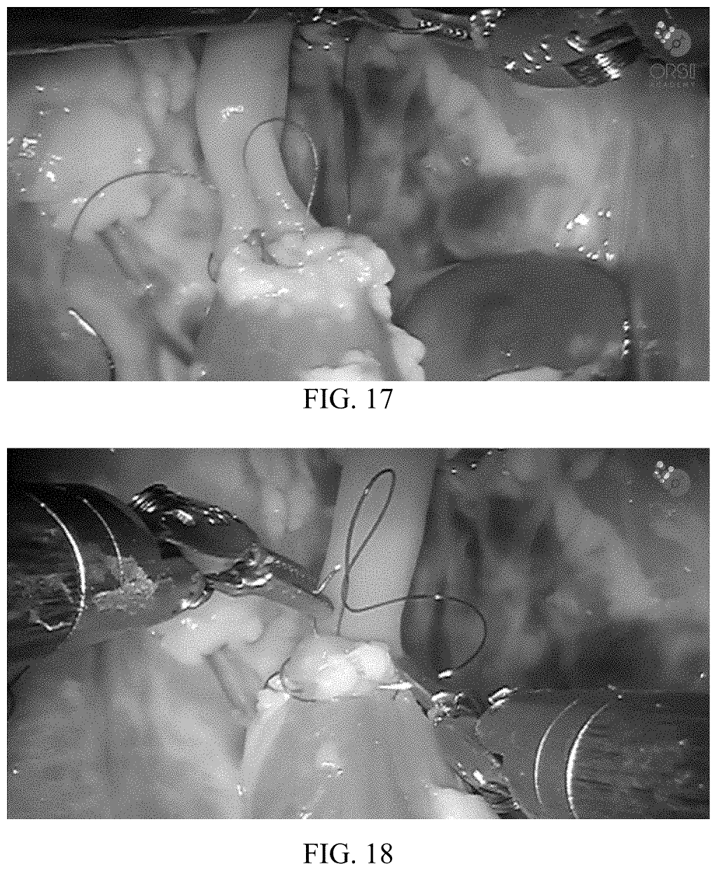

The second step includes the left lateral wall suture, consisting of the bites between the posterior and anterior wall on the left side. The maximum number of bites between the stomach and the cloaca is five (FIG. 16). The second step has the associated errors of (1) conflicts of instruments, (2) missed grasp, (3) instrument not holding the suture nor assisting, (4) tear or injury of the tissue, (5) damage of the suture, (6) incorrect needle grasp, (7) excessive manipulation of the needle, (8) incorrect tip grasp, (9) incorrect bite, (10) incomplete or repeated bite, (11) incorrect suture bite, (12) needle out of view, (13) failure to progress in that specific step of the anastomosis and critical errors of (1) broken needle or suture and (2) catheter fixation during the anastomosis.

The third step includes the right lateral wall suture, consisting of the bites between the posterior and anterior wall on the right side (FIG. 17). The maximum number of bites between the stomach and the cloaca is five. The third step has the associated errors of (1) conflicts of instruments, (2) missed grasp, (3) instrument not holding the suture nor assisting, (4) tear or injury of the tissue, (5) damage of the suture, (6) incorrect needle grasp, (7) excessive manipulation of the needle, (8) incorrect tip grasp, (9) incorrect bite, (10) incomplete or repeated bite, (11) incorrect suture bite, (12) needle out of view, (13) failure to progress in that specific step of the anastomosis and critical errors of (1) broken needle or suture and (2) catheter fixation during the anastomosis.

The fourth step includes the last four needle bites between the stomach and the cloaca on the anterior wall (FIG. 18). The fourth step has the associated errors of (1) conflicts of instruments, (2) missed grasp, (3) instrument not holding the suture nor assisting, (4) tear or injury of the tissue, (5) damage of the suture, (6) incorrect needle grasp, (7) excessive manipulation of the needle, (8) incorrect tip grasp, (9) incorrect bite, (10) incomplete or repeated bite, (11) incorrect suture bite, (12) needle out of view, (13) failure to progress in that specific step of the anastomosis and critical errors of (1) broken needle or suture and (2) catheter fixation during the anastomosis.

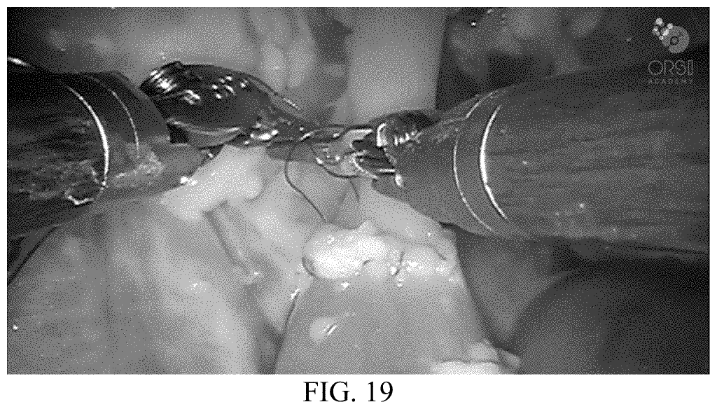

The fifth step includes the suture knotting, consisting of one surgical double knot followed by two single knots out of the cloaca, alternating the direction of the last two throws (FIG. 19). The fifth step has the associated errors of (1) conflicts of instruments, (2) missed grasp, (3) instrument not assisting, (4) tear or injury of the tissue, (5) damage of the suture, (6) incorrect needle grasp, (7) excessive manipulation of the needle, (8) incorrect tip grasp, (9) needle out of view, (10) missed loop, (11) tail looped, (12) loose knot, (13) missed double overhand knot, (14) failure to alternate the direction of the last two throws and the critical error of (1) broken needle or suture.

After the completion of the procedure, the anastomosis is tested through a leakage test. Anastomosis leakage is considered a (1) critical error.

The maximum number of errors allowed in order to meet the defined benchmark is 10 or less. The time limit for procedure completion of suturing and knotting skills training is 40 minutes. Exceeding the time limit determines automatic failure of the task.

2. Skin Dissection Task

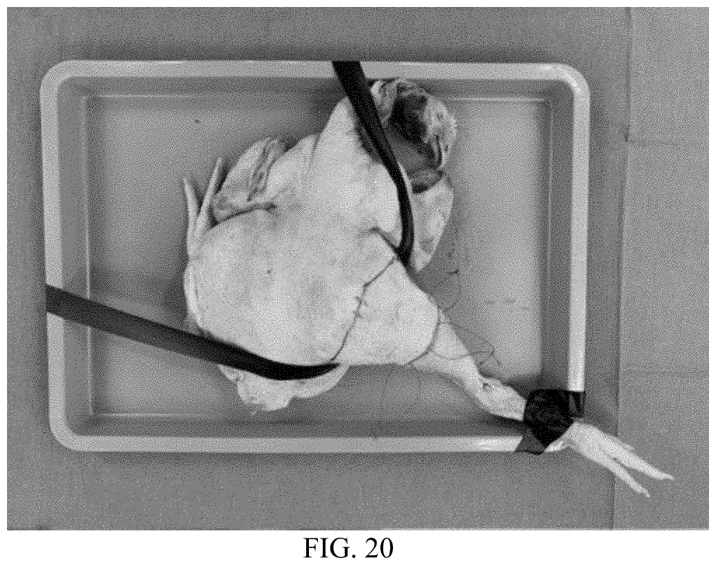

The preparation of the chicken model for this task is standardized. The chicken model is fixed to a tray with tape, with the right leg (left leg for left-handers) dislocated and stretched perpendicular to the body (FIG. 20).

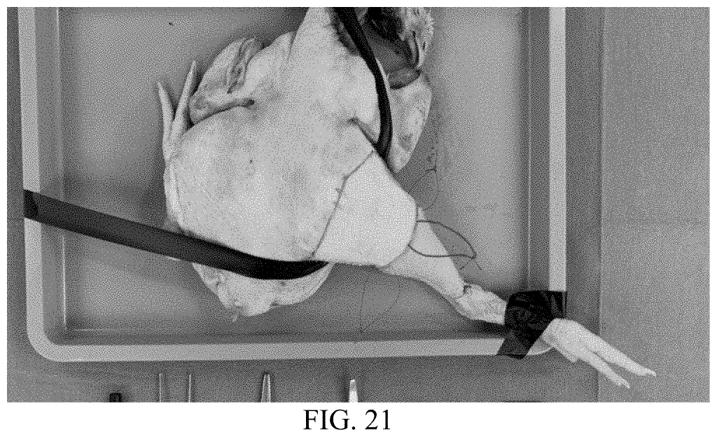

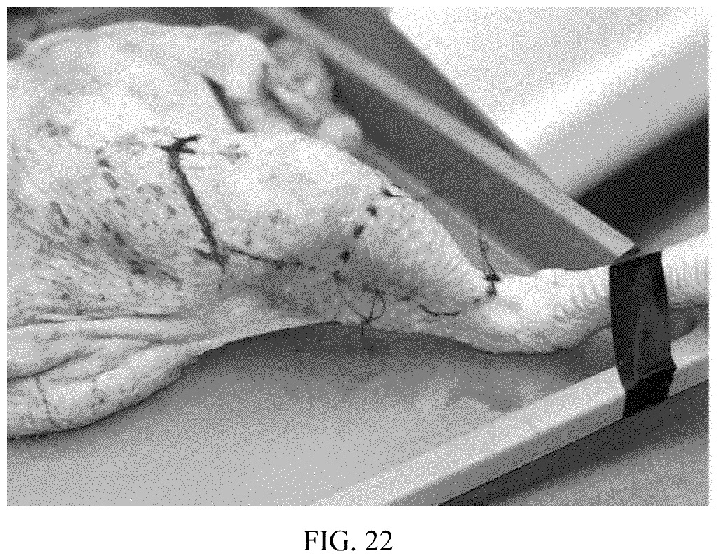

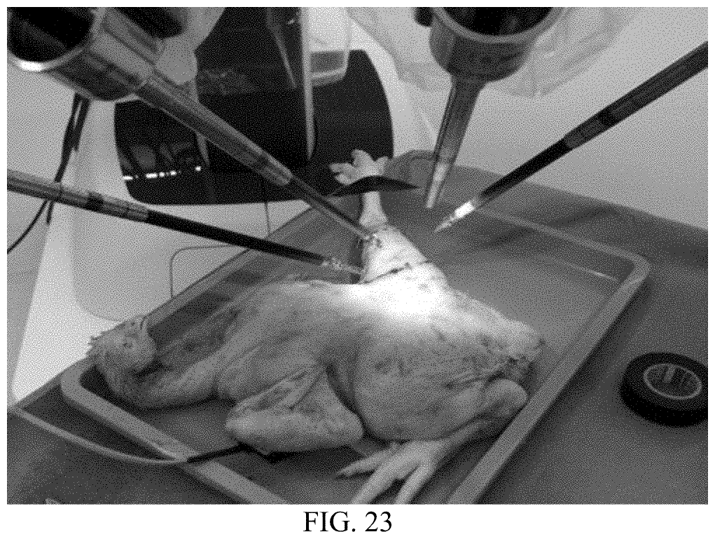

A line drawn in the upper leg represents the beginning of the task. Two small brackets identify the start of the cutting line. A second line drawn at the level of the middle leg, where the skin changes color, represents the transition point from the upper leg to the lower leg. A suture that goes through the skin and the muscle of the chicken allows this transition point to be seen from under the skin. A line at the end of the leg identifies the end of the task and is marked with a suture that can be seen under the skin. The starting line and the middle line delimit the first dissection step, the upper leg step. The middle line and the final line delimit the lower leg step. The lateral limits are visible under the skin due to two sutures (FIG. 21). These limits are visible outside due to two lines that pass in the middle portion of the side faces of the chicken leg (FIG. 22).

The robot is positioned in order to have the camera trocar 10 cm high from the starting line and 15/20 cm away from the target point, the final suture. Robotic trocars must be positioned 8-10 cm apart. In the two trocars positioned close to the camera, the monopolar scissors and the forceps bipolar are inserted. The Prograsp™ is positioned as the fourth arm. The trocars close to the camera are slightly lower than the other two (FIG. 23).

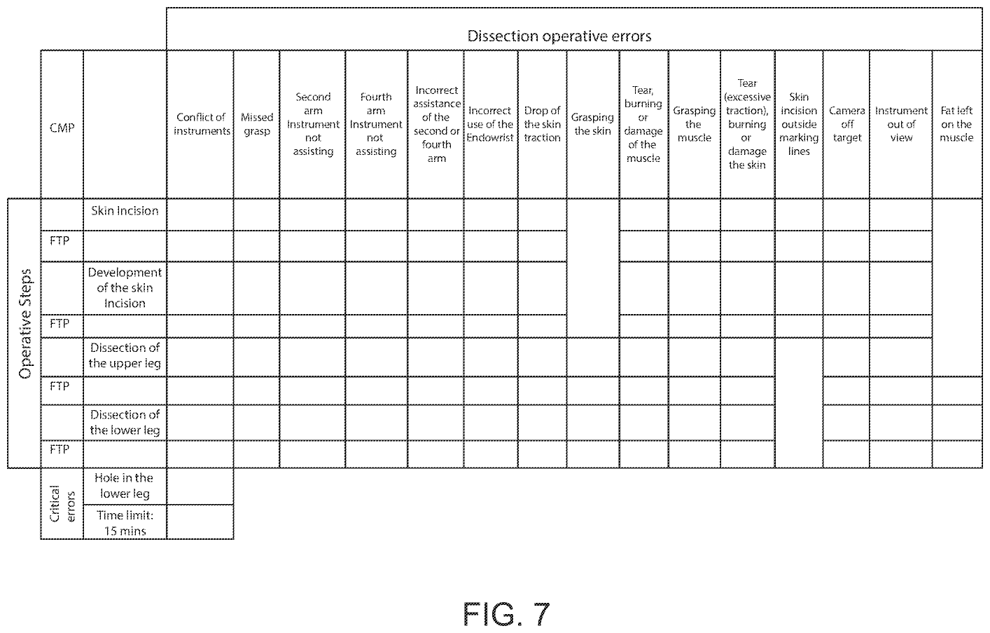

Referring to FIG. 7, the skin dissection task metrics are grouped into four separate steps of the procedure.

The metrics contain a series of related, unambiguously defined, observable procedure steps with specific beginning and ending points to be evaluated. There are also errors and critical errors for the expert to assess throughout the whole procedure. The scoring of the errors starts when the trainee grasps the chicken's skin to begin with the skin incision. The skin dissection task includes four steps, 58 errors and 2 critical errors. The four steps and the errors and critical errors associated with the four steps are listed in the table of FIG. 7, and as further described below.

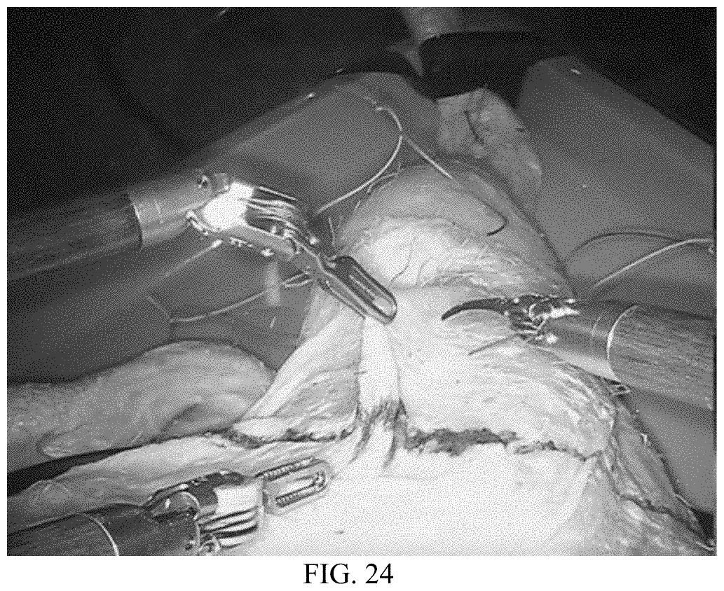

The first step includes the initial skin incision, made between the two central marking lines (1-2 cm wide). During this step, the use of diathermic energy is mandatory. The fourth and the second arm should be used to stretch the two sides of the incision line (FIG. 24). The first step has the associated errors of (1) conflicts of instruments, (2) missed grasp, (3) second arm instrument not assisting, (4) fourth arm instrument not assisting, (5) incorrect assistance of the second or fourth arm, (6) incorrect use of the EndoWrist, (7) drop of the skin traction, (8) tear, burning or damage of the muscle, (9) grasping of the muscle, (10) tear, burning or damage of the skin, (11) skin incision outside the marking line, (12) camera off target, (13) instrument out of view, (14) fail to progress in the specific step of the procedure and critical error of (1) exceeding of the time limit.

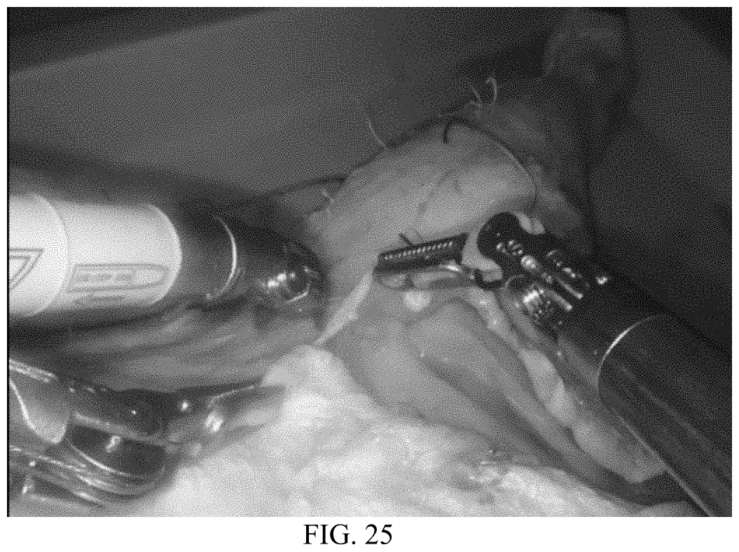

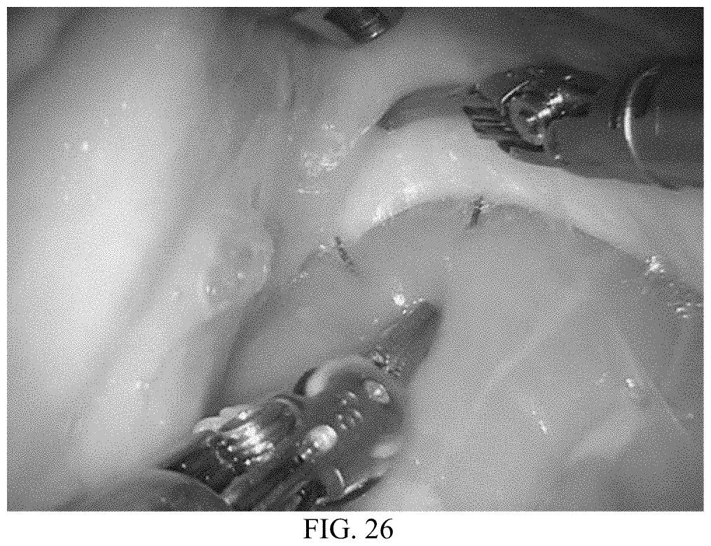

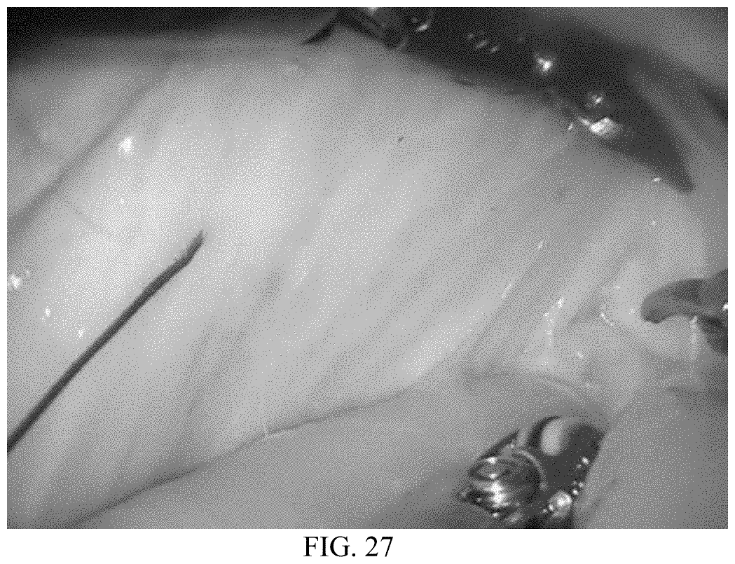

The second step consists of the development of the initial skin incision. The skin incision must be developed within the two lateral marking lines (total length 10-12 cm). Skin incisions outside the marking line are not allowed. Coagulation and grasping of the skin with robotic instruments are allowed (FIG. 25). The second step has the associated errors of (1) conflicts of instruments, (2) missed grasp, (3) second arm instrument not assisting, (4) fourth arm instrument not assisting, (5) incorrect assistance of the second or fourth arm, (6) incorrect use of the EndoWrist, (7) drop of the skin traction, (8) tear, burning or damage of the muscle, (9) grasping of the muscle, (10) tear, burning or damage of the skin, (11) skin incision outside the marking line, (12) camera off target, (13) instrument out of view, (14) failure to progress in the specific step of the procedure and critical error of (1) exceeding of the time limit.

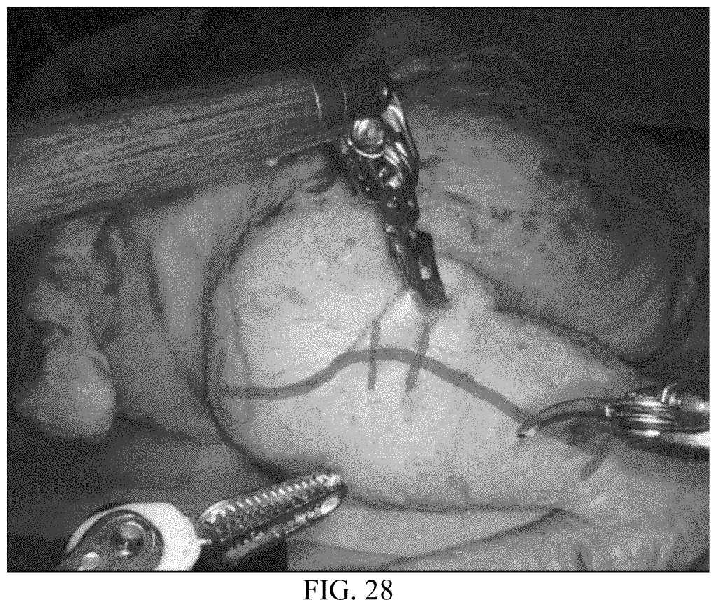

The third step consists of the dissection of the upper leg. The starting point of this step is the skin marking. By using the principles of traction and countertraction, using the assistance of the third arm and the EndoWrist, the avascular place between the skin and the underlying muscle is bluntly developed. The dissection is that of the leg. During the dissection, the fat laying on the muscle must be completely removed by blunt and sharp dissection. Grasping of the fat is allowed. The step is completed when the two lateral stitches are visualized, and the first transversal stitch is cut. Use of diathermic energy is not allowed during this step (FIG. 26). The third step has the associated errors of (1) conflicts of instruments, (2) missed grasp, (3) second arm instrument not assisting, (4) fourth arm instrument not assisting, (5) incorrect assistance of the second or fourth arm, (6) incorrect use of the EndoWrist, (7) drop of the skin traction, (8) grasping of the skin, (9) tear, burning or damage of the muscle, (10) grasping of the muscle, (11) tear, burning or damage of the skin, (12) camera off target, (13) instrument out of view, (14) fat left on the muscle, (15) failure to progress in the specific step of the procedure and critical error of (1) exceeding of the time limit.

The fourth step consists of the dissection of the lower leg. The starting point of this step is the elbow bone, identified by the first transversal stitch. By using the principles of traction and countertraction, using the assistance of the third arm and the EndoWrist, the avascular place between the skin and the underlying muscle is bluntly developed. The dissection is that of the leg. During the dissection, the fat laying on the muscle must be completely removed by blunt and sharp dissection. Grasping of the fat is allowed. The step is completed when the second transversal stitch is under vision. Use of diathermic energy is not allowed during this step (FIG. 27). The fourth step has the associated errors of (1) conflicts of instruments, (2) missed grasp, (3) second arm instrument not assisting, (4) fourth arm instrument not assisting, (5) incorrect assistance of the second or fourth arm, (6) incorrect use of the EndoWrist, (7) drop of the skin traction, (8) grasping of the skin, (9) tear, burning or damage of the muscle, (10) grasping of the muscle, (11) tear, burning or damage of the skin, (12) camera off target, (13) instrument out of view, (14) fat left on the muscle, (15) failure to progress in the specific step of the procedure and critical errors of (1) exceeding of the time limit and (2) skin tear/hole in the lower leg skin.

The maximum number of errors allowed in order to score the defined benchmark is 6. The time limit for procedure completion of the skin dissection task is 15 minutes. Exceeding the time limit is defined as a critical error.

3. Dissection and Coagulation Task

The preparation of the chicken model for this task is standardized. The chicken model is placed and fixed to a tray with tape, the chicken model laying on the breast and with the right lower leg (left leg for left-handers) folded against the body. A line is drawn on the lower leg between the two joints, representing the start of the task. Two small brackets identified the start of the cutting line (FIG. 28).

The robot is positioned in order to have the camera trocar 10 cm high from the starting line. Robotic trocars must be positioned at a distance of 8-10 cm apart. In the two trocars positioned close to the camera the monopolar scissors and the forceps bipolar are inserted. The Prograsp™ is positioned as the fourth arm. A large needle driver is used in one of the steps of the task in place of the monopolar scissor. The trocars close to the camera are slightly lower than the other two.

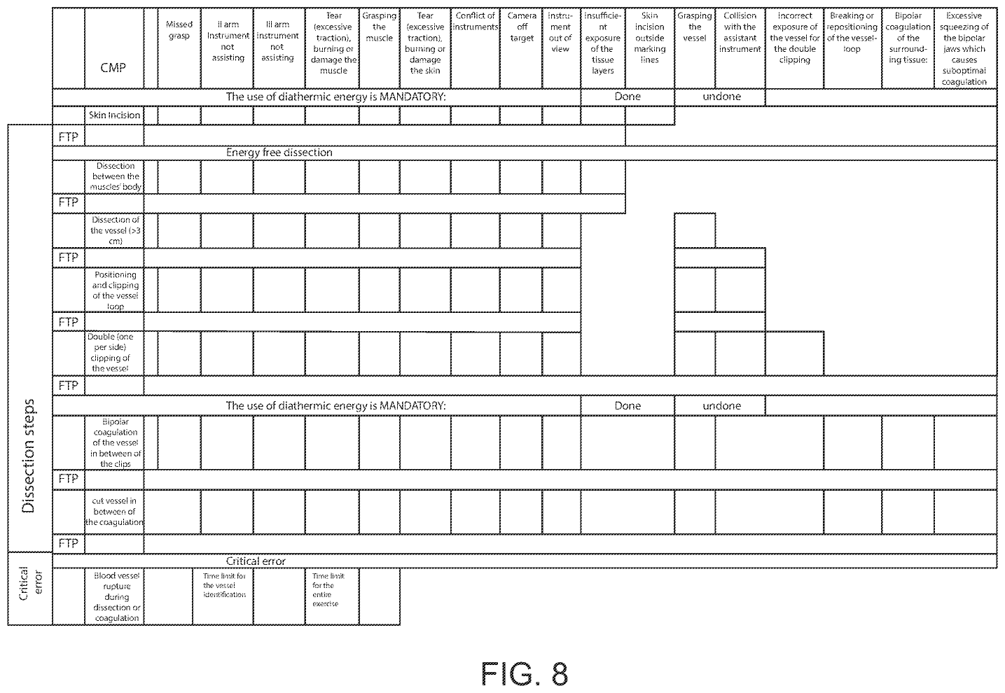

Referring to FIG. 8, the dissection and coagulation task metrics are grouped into seven separate steps of the procedure.

The metrics contain a series of related, unambiguously defined, observable procedure steps with specific beginning and ending points to be evaluated. There are also errors and critical errors for the expert to assess throughout the whole procedure. The scoring of the errors starts when the trainee grasps the chicken's skin to begin with the skin incision. Dissection and coagulation task include seven steps, 85 errors (3 unique) and 3 critical errors. The seven steps and the errors and critical errors associated with the four steps are listed in the table of FIG. 8, and as further described below.

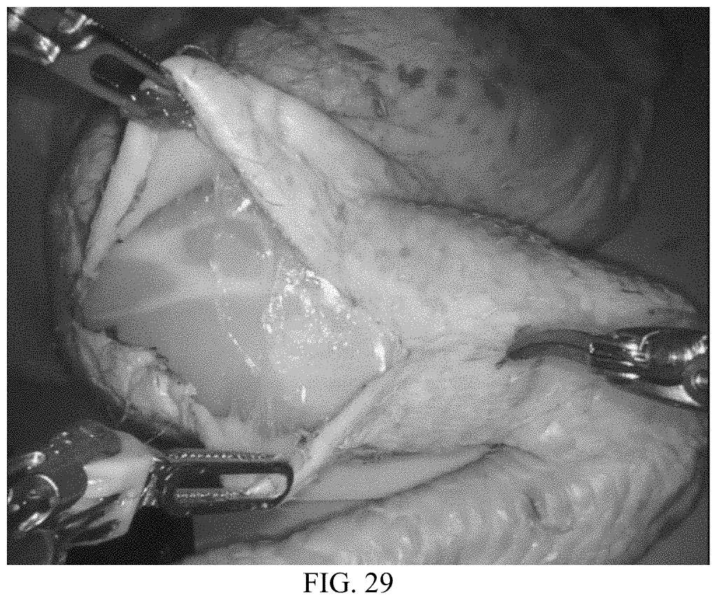

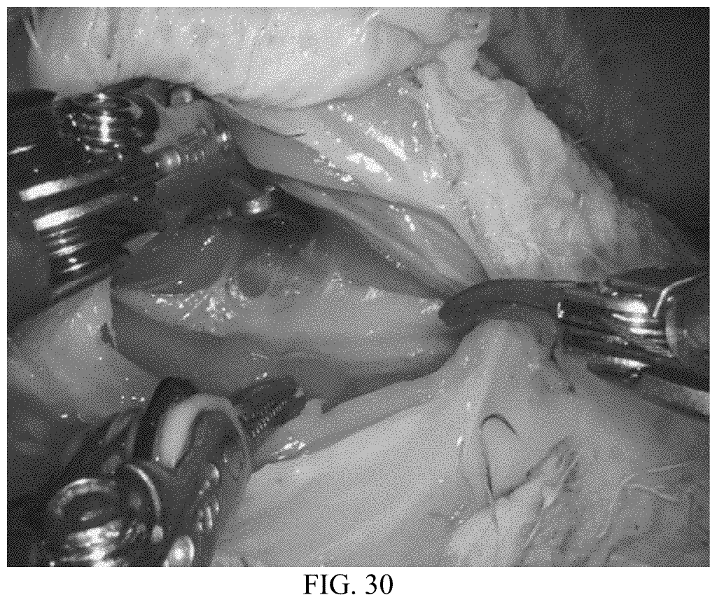

The first step includes the incision of the skin of the chicken model, made within the drawn marking line. The use of diathermic energy is mandatory to complete the step and is assessed in the assessment table. The fourth and the second arm should be used to stretch the two sides of the incision line (FIG. 29). The first step has the associated errors of (1) missed grasp, (2) second arm instrument not assisting, (3) third arm instrument not assisting, (4) tear, burning or damage of the muscle, (5) grasping of the muscle, (6) tear, burning or damage of the skin, (7) conflicts of instruments, (8) camera off target, (9) instrument out of view, (10) skin incision outside marking line, (11) failure to progress in the specific step of the procedure and critical error of (1) exceeding of the time limit for the entire exercise completion.

The second step includes the dissection between the muscles' body. The incision of the muscle is started incising without the use of diathermic energy along the visible tendon's line. Subsequent sharp and blunt dissection between the muscular bellies is conducted until the vessel, located posteriorly and above the bone, is identified. Grasping of tendons is allowed. Grasping of the muscle is forbidden. The use of diathermic energy is also forbidden. The use of the fourth arm is recommended (FIG. 30). The second step has the associated errors of (1) missed grasp, (2) second arm instrument not assisting, (3) third arm instrument not assisting, (4) tear, burning or damage of the muscle, (5) grasping of the muscle, (6) tear, burning or damage of the skin, (7) conflicts of instruments, (8) camera off target, (9) instrument out of view, (10) insufficient exposure of the tissue layers, (11) failure to progress in the specific step of the procedure and critical errors of (1) exceeding of the time limit for the vessel identification and (2) exceeding of the time limit for the entire exercise completion.

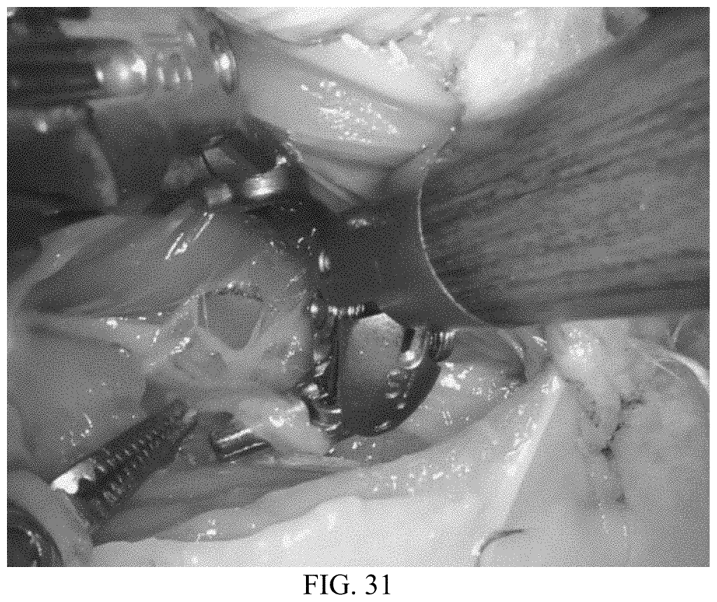

The third step includes the dissection and isolation of the vessel (>3 cm). This phase starts when the trainee has identified the vessel and begins to selectively separate it from the surrounding tissues. The vessel must be completely freed from the surrounding tissues for a length of 3 cm (approximately one and a half times the length of the Prograsp jaws). Grasping of the vessel is prohibited. The use of diathermic energy is forbidden. The use of the fourth arm is recommended (FIG. 31). The third step has the associated errors of (1) missed grasp, (2) second arm instrument not assisting, (3) third arm instrument not assisting, (4) tear, burning or damage of the muscle, (5) grasping of the muscle, (6) tear, burning or damage of the skin, (7) conflicts of instruments, (8) camera off target, (9) instrument out of view, (10) grasping of the vessel, (11) failure to progress in the specific step of the procedure and critical errors of (1) blood vessel rupture during dissection or coagulation and (2) exceeding of the time limit for the entire exercise completion.

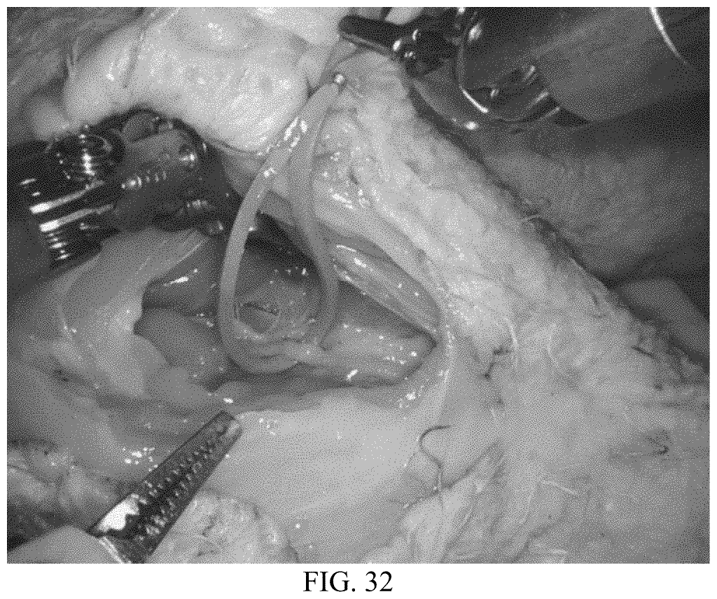

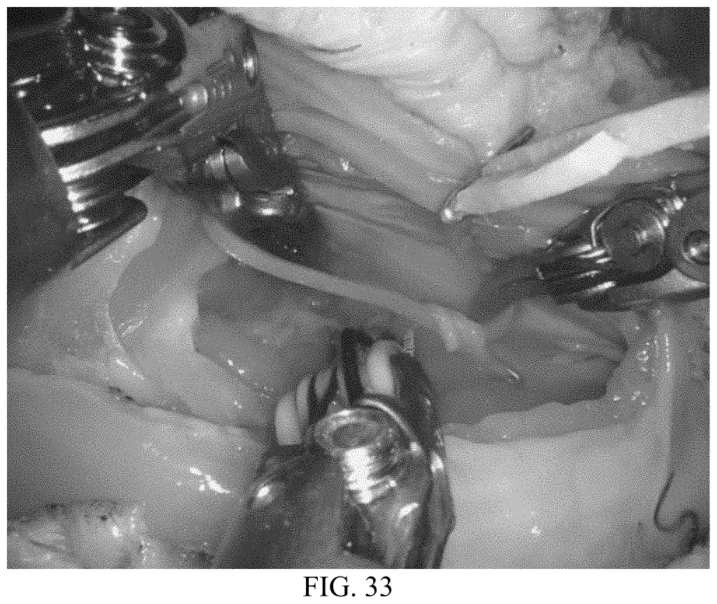

The fourth step includes the positioning and clipping of the vessel loop around the isolated vessel. A 10 cm vessel loop must be placed. The monopolar scissor must be substituted with a large needle driver to facilitate the procedure. A clip must be positioned to join the two ends of the vessel loop. The assistant instrument must come between the camera trocar and the first right trocar. The vessel loop can both be given to the trainee or placed following the trainee instructions. Grasping of the vessel is prohibited. The use of diathermic energy is forbidden. The use of the fourth arm is recommended (FIG. 32). The fourth step has the associated errors of (1) missed grasp, (2) second arm instrument not assisting, (3) third arm instrument not assisting, (4) tear, burning or damage of the muscle, (5) grasping of the muscle, (6) tear, burning or damage of the skin, (7) conflicts of instruments, (8) camera off target, (9) instrument out of view, (10) grasping of the vessel, (11) collision with the assistant's instruments, (12) failure to progress in the specific step of the procedure and critical errors of (1) blood vessel rupture during dissection or coagulation and (2) exceeding of the time limit for the entire exercise completion.

The fifth step includes the double (one per side) clipping of the vessel. Two clips must be placed on the sides of the dissected vessel. The trainee guides the assistant for the correct positioning. The assistant instrument comes between the camera trocar and the first right trocar. Grasping of the vessel is prohibited (FIG. 33). The fifth step has the associated errors of (1) missed grasp, (2) second arm instrument not assisting, (3) third arm instrument not assisting, (4) tear, burning or damage of the muscle, (5) grasping of the muscle, (6) tear, burning or damage of the skin, (7) conflicts of instruments, (8) camera off target, (9) instrument out of view, (10) grasping of the vessel, (11) collision with the assistant's instruments, (12) incorrect exposure of the vessel for double clipping, (13) failure to progress in the specific step of the procedure and critical errors of (1) blood vessel rupture during dissection or coagulation and (2) exceeding of the time limit for the entire exercise completion.

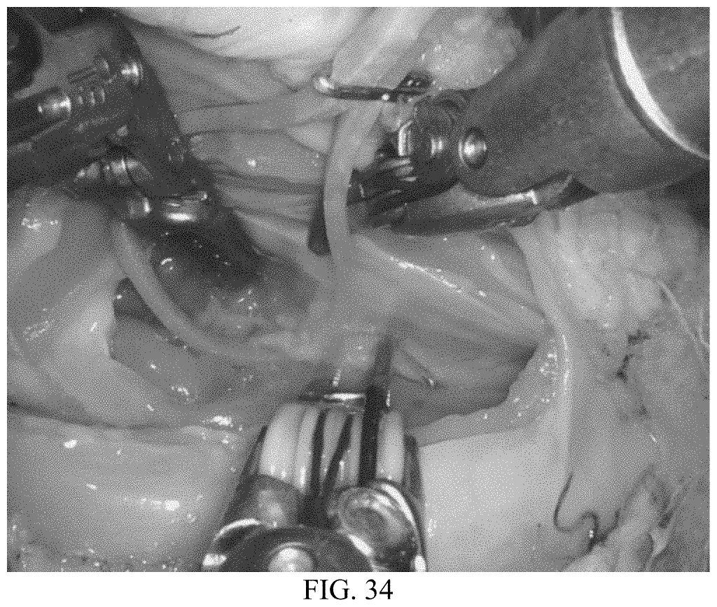

The sixth step includes the bipolar coagulation of the vessel in between the clips. The bipolar coagulation must be applied near and medially to each clip. Grasping of the vessel is prohibited. The use of bipolar coagulation is mandatory in order to complete the step (FIG. 34). The sixth step has the associated errors of (1) missed grasp, (2) second arm instrument not assisting, (3) third arm instrument not assisting, (4) tear, burning or damage of the muscle, (5) grasping of the muscle, (6) tear, burning or damage of the skin, (7) conflicts of instruments, (8) camera off target, (9) instrument out of view, (10) grasping of the vessel, (11) breaking or repositioning of the vessel loop, (12) bipolar coagulation of the surrounding tissues, (13) excessive squeezing of the bipolar jaws which causes suboptimal coagulation, (14) failure to progress in the specific step of the procedure and critical errors of (1) blood vessel rupture during dissection or coagulation and (2) exceeding of the time limit for the entire exercise completion.

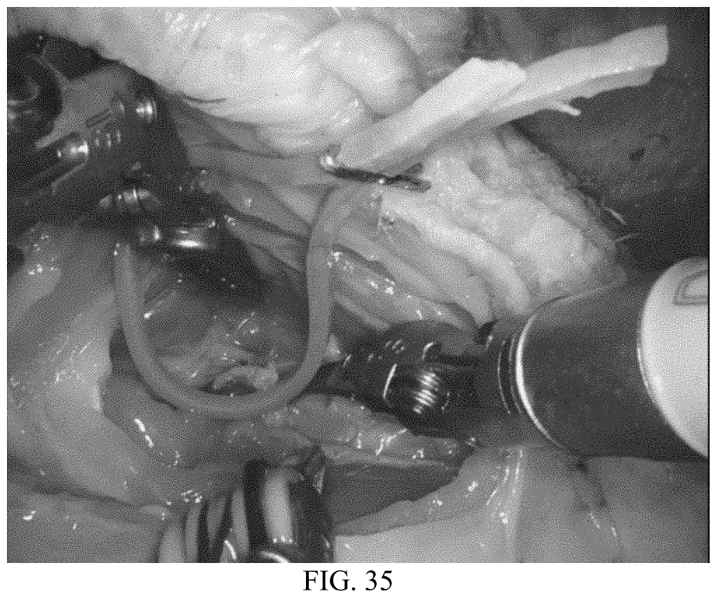

The seventh step includes cutting the vessel in between the clips and in the middle of the coagulated tissue. Grasping of the vessel is prohibited. The use of bipolar coagulation is forbidden in order to complete the step (FIG. 35). The sixth step has the associated errors of (1) missed grasp, (2) second arm instrument not assisting, (3) third arm instrument not assisting, (4) tear, burning or damage of the muscle, (5) grasping of the muscle, (6) tear, burning or damage of the skin, (7) conflicts of instruments, (8) camera off target, (9) instrument out of view, (10) grasping of the vessel, (11) breaking or repositioning of the vessel loop, (12) bipolar coagulation of the surrounding tissues, (13) failure to progress in the specific step of the procedure and critical errors of (1) blood vessel rupture during dissection or coagulation and (2) exceeding of the time limit for the entire exercise completion.

The maximum number of errors allowed in order to score the defined benchmark is 6. The time limit for procedure completion of the dissection and coagulation task is 15 minutes. Exceeding the time limit is defined as a critical error.

It should be understood that the invention is not limited in its application to the details of construction and arrangements of the components set forth herein. The invention is capable of other robotic surgical embodiments and of being practiced or carried out in various ways. Variations and modifications of the foregoing are within the scope of the present invention. It also being understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

We claim:

1. A method of training a trainee for a robotic-assisted procedure, the method comprising the steps of:
a) recording a video of the trainee performing a task on a nonliving chicken model;
b) reviewing the video of the trainee performing the task on the non-living chicken model;
c) determining whether or not observable, explicitly defined and validated set of performance metrics for evaluation are performed by the trainee, wherein the set of metrics are at least one of a discrete performance element, an order in which specific operative steps should be accomplished, and instruments and the manner in which they should be used;
d) inputting a first indication if a metric of the set of metrics is performed and a second indication if the metric is not performed into an evaluation report; and
e) providing a summary report based upon the evaluation report of the trainee's performance.

2. The method of claim 1 wherein the trainee performs the task using controls mechanically manipulating corresponding robotic arms of a robot interacting with the non-living chicken model.

3. The method of claim 2 wherein the task includes a suturing and knot tying task wherein the non-living chicken model is prepared by removing all abdominal organs except for 6-8 cm of a cloaca and a stomach.

4. The method of claim 3 wherein the suturing and knot tying task includes at least one of the following procedure steps: suturing of a posterior wall; suturing of a left lateral wall; suturing of a right lateral wall; suturing of an anterior wall; and knotting.

5. The method of claim 4 wherein the observable, explicitly defined and validated performance metrics further include a deviation from optimal performance (or errors), the deviation is at least one of the following: a conflict of instruments; a missed grasp; an instrument not assisting; a tear or injury of tissue; damage of a suture; an incorrect needle grasp; excessive manipulation; incorrect tip grasp; incorrect bite; incomplete or repeated bite; incorrect suture bite; needle out of view; missed loop; tailed loop; loose knot; missed double overhand knot; failure to alternate a direction of a last two throws; and failure to progress.

6. The method of claim 5 wherein the deviation includes at least one of the following observable, explicitly defined and validated performance critical errors: an anastomosis leakage; a broken needle or suture; and a catheter fixation during anastomosis.

7. The method of claim 2 wherein the task includes a skin dissection task wherein the non-living chicken model is prepared by fixing the nonliving chicken model to a tray with tape, with a leg of the chicken dislocated and stretched perpendicular to a body of the nonliving chicken model.

8. The method of claim 7 wherein the skin dissection task includes performing at least one of the following observable, explicitly defined and validated performance metric-steps: a skin incision; development of a skin incision; a dissection of an upper leg; and a dissection of a lower leg.

9. The method of claim 8 wherein the metrics further include a observable, explicitly defined and validated deviations from optimal performance, the deviation is at least one of the following: a conflict of instruments; a missed grasp; a second arm instrument not assisting; a fourth arm instrument not assisting; an incorrect assistance of the second or fourth arm; an incorrect use of an Endowrist; a drop of a skin traction; grasping skin; a tear (excessive traction), burning or damage of a muscle; a grasping of muscle; a tear (excessive traction), burning or damage skin; a skin incision outside marking lines; camera off target; instrument out of view; and fat left on muscle.

10. The method of claim 9 wherein the deviation includes at least one of the following observable, explicitly defined and validated set of critical errors: a hole in a lower leg; and exceeding a time limit for the skin dissection task.

11. The method of claim 2 wherein the task includes a dissection and coagulation task wherein the non-living chicken model is prepared by fixing the nonliving chicken model to a tray with tape and laying the non-living chicken model breast down with a leg folded against a body of the nonliving chicken model.

12. The method of claim 11 wherein the dissection and coagulation task includes performing at least one of the following observable, explicitly defined and validated metric steps: a skin incision; a dissection between muscles' body; a dissection of a vessel (>3 cm); a positioning and clipping of a vessel loop; a double (one per side) clipping of the vessel; a bipolar coagulation of the vessel in between clips; and a vessel cut in between the bipolar coagulation.

13. The method of claim 12 wherein the metrics further include a observable, explicitly defined and validated set of performance deviations from optimal performance (i.e., performance errors), the deviation is at least one of the following: a missed grasp; a second arm instrument not assisting; a third arm instrument not assisting; a tear (excessive traction), a burning or damage of a muscle; a grasping of muscle; a tear (excessive traction), burning or damage of skin; a conflict of instruments; camera off target; an instrument out of view; an insufficient exposure of tissue layers; a skin incision outside marking lines; grasping a vessel; a collision with an assistant instrument; an incorrect exposure of the vessel for double clipping; a breaking or repositioning of a vessel loop; a bipolar coagulation of surrounding tissue; and an excessive squeezing of bipolar jaws causing suboptimal coagulation.

14. The method of claim 13 wherein the deviation includes at least one of the following observable, explicitly defined and validated set of critical errors: a blood vessel rupture during dissection or coagulation; exceeding a time limit for vessel identification; and exceeding a time limit for the dissection and coagulation task.

15. The method of claim 2 wherein the summary report includes an average score for number of steps of the procedure completed, the number of procedure errors made and what they were, and the number of procedure critical errors made and what they were.

16. The method of claim 2 wherein the summary report includes a total time to perform the procedure and whether the performance benchmarks was met or not.
